(19) United States
(12) Patent Application Publication
(10) Pub. No.: US 2026/0069765 A1
(43) Pub. Date: Mar. 12, 2026

(54) CARTRIDGE ASSEMBLY WITH INTEGRATED INFUSION CATHETER AND CGM SENSOR SUBASSEMBLY

(71) Applicant: AITA BIO INC., New York, NY (US)

(72) Inventors: Dilan Casanovas Mack, New York, NY (US); Russell Cole, River Vale, NJ (US)

(21) Appl. No.: 19/106,581

(22) PCT Filed: Sep. 16, 2023
(86) PCT No.: PCT/US23/32967

§ 371 Date: Feb. 25, 2025

Related U.S. Application Data
(60) Provisional application No. 63/414,091, filed on Oct. 7, 2022.

(51) Int. Cl. A61M 5/142; A61M 5/158

(52) U.S. Cl. CPC A61M 5/14248; A61M 5/158; A61M 2005/14252; A61M 2005/1585; A61M 2202/0007; A61M 2202/0468; A61M 2205/3303

(57) ABSTRACT

An infusion system is disclosed comprising: a device for delivering insulin into tissue of a user; and a cartridge assembly including an integrated infusion catheter and CGM sensor subassembly and an introducer needle for introducing the integrated infusion catheter and CGM sensor subassembly into the tissue of the user, wherein the cartridge assembly configured to move from (a) first position, wherein the integrated infusion catheter and CGM sensor subassembly is in a first position above the tissue of the user to (b) a second position, wherein the integrated infusion catheter and CGM sensor subassembly is in a deployed position inserted into tissue of the user.

### CROSS REFERENCE TO RELATED APPLICATIONS

This application claims priority to U.S. provisional application number 63/414,091, filed Oct. 7, 2022, entitled “Cartridge Assembly With Integrated Infusion Catheter and CGM Sensor Subassembly” which is incorporated by reference herein.

### FIELD OF THE INVENTION

The present invention relates to a cartridge assembly with an integrated infusion catheter and CGM sensor subassembly.

### BACKGROUND OF THE INVENTION

Insulin pumps help people with diabetes to conveniently manage their blood sugar. These devices deliver insulin at specific times. Insulin patch pumps or pods are one type of insulin pump. The pods are wearable devices that adhere to the skin of a user using an adhesive patch. The pods deliver insulin from a chamber and internal cannula based on separately acquired continuous glucose monitoring (CGM) sensor readings.

It would be advantageous to provide improvements to insulin pumps described above.

### SUMMARY OF THE INVENTION

A cartridge assembly is disclosed with an integrated infusion catheter and CGM sensor subassembly.

In accordance with an embodiment of the present disclosure, An infusion system comprising: a device for delivering fluid into tissue of a user; an infusion needle; an analyte sensor; an introducer needle for introducing the infusion catheter and the analyte sensor into the tissue of the user; and a carrier configured to carry the introducer needle along with the infusion catheter and analyte sensor, wherein the carrier is configured to move from (a) first position, wherein the introducer needle, the. infusion catheter and the analyte sensor are in a retracted position above the tissue of the user to (b) a second position, wherein the introducer needle, infusion catheter and analyte sensor are in a deployed position inserted into tissue of the user.

In accordance with another embodiment of the disclosure, an infusion system comprising: a device for delivering insulin into tissue of a user; and a cartridge assembly including a subassembly for supporting an infusion catheter and a CGM sensor, the subassembly including: the infusion catheter; the CGM sensor; and an introducer needle for introducing the subassembly for supporting the infusion catheter and the CGM sensor into the tissue of the user, wherein the cartridge assembly configured to move from (a) first position, wherein the subassembly is in a retracted position above the tissue of the user to (b) a second position, wherein the subassembly is in a deployed position inserted into tissue of the user.

In accordance with another embodiment of the present disclosure, an infusion system comprising: a device for delivering insulin into tissue of a user; and a cartridge assembly including an integrated infusion catheter and CGM sensor subassembly and an introducer needle for introducing the integrated infusion catheter and CGM sensor subassembly into the tissue of the user, wherein the cartridge assembly configured to move from (a) first position, wherein the integrated infusion catheter and CGM sensor subassembly is in a first position above the tissue of the user to (b) a second position, wherein the integrated infusion catheter and CGM sensor subassembly is in a deployed position inserted into tissue of the user.

### BRIEF DESCRIPTION OF DRAWINGS

FIG. 1 depicts an example cartridge assembly of a device for delivering insulin.

FIG. 2 depicts the cartridge assembly shown in FIG. 1 after insertion of CGM sensor but with introducer needle removed.

FIG. 3 depicts a cross sectional view of the infusion system with the cartridge of FIG. 1 inside detachable activation mechanism in a pre-activation configuration.

FIG. 4 depicts a side view of infusion catheter and CGM sensor subassembly including infusion catheter, introducer needle and sensor in a pre-activation configuration.

FIG. 5 depicts a close view of infusion system through an opening in a baseplate of delivery device 300 through which an introducer needle and infusion catheter pass during insertion.

FIG. 6 depicts the infusion system after insertion of integrated infusion catheter and CGM sensor subassembly by the cartridge deployment (in advance state), but before the introducer needle has been retracted.

FIG. 7 depicts a side cross-sectional view of the delivery device in wearable deployed configuration on a user.

FIG. 8 depicts a side view of the delivery device in a deployed configuration (applied to a user), but with infusion catheter and CGM sensor shown.

FIG. 9 depicts a side view of another example integrated infusion catheter and CGM sensor subassembly.

FIG. 10 depicts another example integrated infusion catheter and CGM sensor subassembly.

FIG. 11 depicts an example H shaped introducer needle of an integrated infusion and CGM sensor subassembly.

FIG. 12 depicts another example integrated infusion catheter and CGM sensor subassembly.

FIG. 13 depicts the example subassembly in FIG. 12 but from a view normal to the primary axis of the catheter.

FIG. 14 depicts a block diagram of example components of the device for delivering insulin and cartridge assembly of the infusion system.

### DETAILED DESCRIPTION OF THE INVENTION

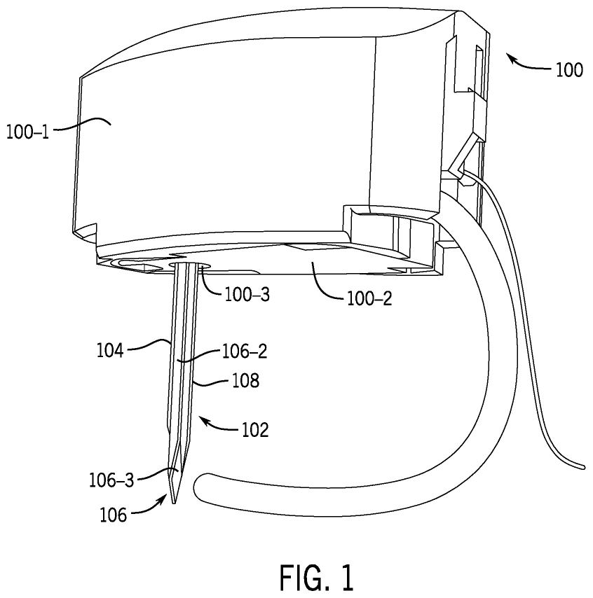

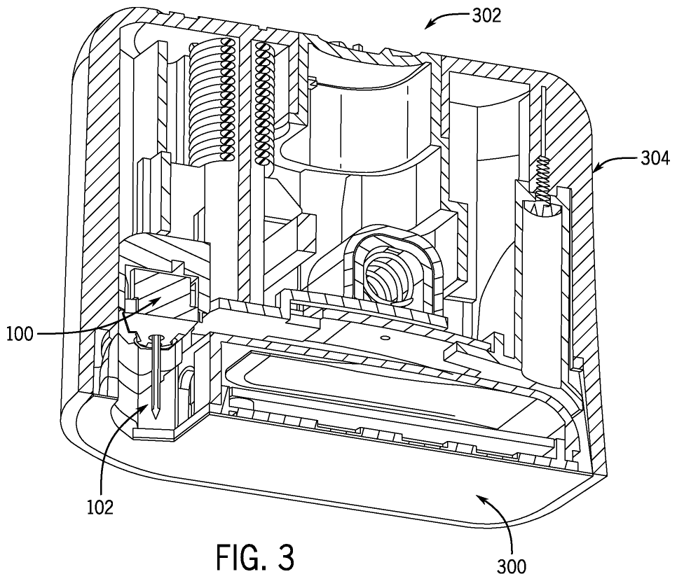

FIG. 1 depicts an example cartridge assembly 100 of device 300 for delivering insulin (shown in FIG. 3) or other medical fluids (also referred to as medication or medicament) such as such as small molecule pharmaceutical solutions, large molecule or protein drug solutions, saline solutions, blood or other fluids known to those skilled in the art.

Cartridge assembly 100 incorporates an integrated infusion catheter and continuous glucose monitoring (CGM) sensor subassembly 102 as described in more detail below. Device 300 (also referred to as a delivery device 300) is component of infusion system 302 (FIG. 3) for infusing insulin or other fluid medication into a user (patient). Subassembly 102 is considered be integrated in that both infusion catheter and CGM sensor are assembled and inserted together using an introducer needle. (In this respect however, infusion catheter and CGM sensor may penetrate tissue at the same time or different times when inserted together depending on assembly.) This as described in more detail below.

Cartridge assembly 100 is an example of a vehicle or carrier to carry and introduce/insert infusion catheter and CGM sensor subassembly 102 into tissue of the user. Those skilled in the art know that any other vehicle maybe used to insert the subassembly 102. (Subassembly 102 may also be referred to as subassembly 102 for supporting an infusion catheter and CGM sensor.)

Infusion system 302 also includes detachable activation mechanism 304 that is configured to insert indwelling integrated infusion catheter and CGM sensor subassembly 102 into the subcutaneous tissue of a user's skin as a single insertion site normal to the user's skin. Specifically, detachable activation mechanism 304 is configured to drive cartridge assembly 100 from a telescoping position on the top surface of device 300, wherein integrated infusion needle and CGM sensor subassembly 102 is in a retracted position to (2) an advanced position within device 300, wherein subassembly 102 is in a deployed position embedded into the subcutaneous layer of the user's tissue (after insertion). Detachable activation mechanism 304 is further configured to insert integrated infusion catheter and CGM sensor subassembly 102 in a single insertion site normal to a user's skin or at other desired angles known to those skilled in the art. This is described in more detail below.

Device 300 is a wearable apparatus, system or pod for diabetes management in which continuous glucose monitoring (CGM), insulin delivery and control functionality are provided to ensure insulin is delivered at very precise rates and has the capability of detecting occlusions in real time. In operation, device 300 is applied by opening a sterile packaging, filling the reservoir with insulin, priming the fluid path, removing the adhesive backing, sticking infusion system 302 to the desired body location, pushing a button assembly, removing and disposing the detachable activation mechanism 304. Removing detachable activation mechanism 304 components that are necessary for device 300 activation and needle insertion, but not required for infusion or sensing offers the benefit of a smaller, lower profile, more comfortable and discrete wearable device 300 for the user. This is described in more detail below.

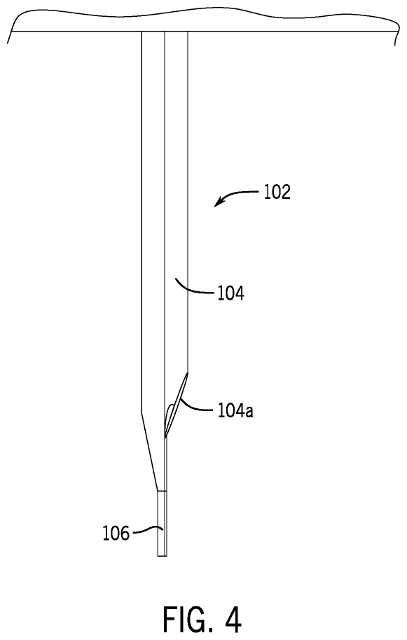

As described in detail below, infusion catheter and CGM sensor subassembly 102 includes infusion catheter 104, introducer needle 106 (removable), and continuous glucose monitoring (CGM) sensor 108 (as one type of analyte sensor). The introducer needle 106 may be removed manually through a hole in the cartridge assembly 100 or automatically by mechanical apparatus as known to those skilled in the art. Introducer needle 106 includes retention element including dual parallel walls 106-1,106-2 that extend from back wall 106-3. Walls 106-1,106-2,106-3 are configured as a square trough shape to receive and hold CGM sensor 108. Infusion catheter 104 extends along the reverse side of back wall 106-3. End 104a of infusion catheter 104 (shown for example in FIG. 4) has a bevel planar shape so that end 104a of infusion catheter 104 is configured and functions as a needle. This is described below with respect to FIG. 4. Introducer needle 106 is preferably constructed of steel but it may be any rigid material known to those skilled in the art. Infusion catheter 104 may be steel or plastic (including PEEK). CGM sensor 108 is constructed of many materials as known to those skilled in the art.

Device 300 incorporates, among other elements (as described below), a micropump as known to those skilled in the art that can be used for pumping fluid, valves used for regulating flow, actuators used for moving or controlling the micropump and valves and/or sensors used for sensing pressure and/or flow. The micropump may be used to infuse the insulin or other fluidic medication to the user (patient). Medication may include small molecule pharmaceutical solutions, large molecule or protein drug solutions, saline solutions, blood or other fluids known to those skilled in the art. Insulin is an example of fluid that is described in this application. However, micropump may be used in other environments known to those skilled in the art.

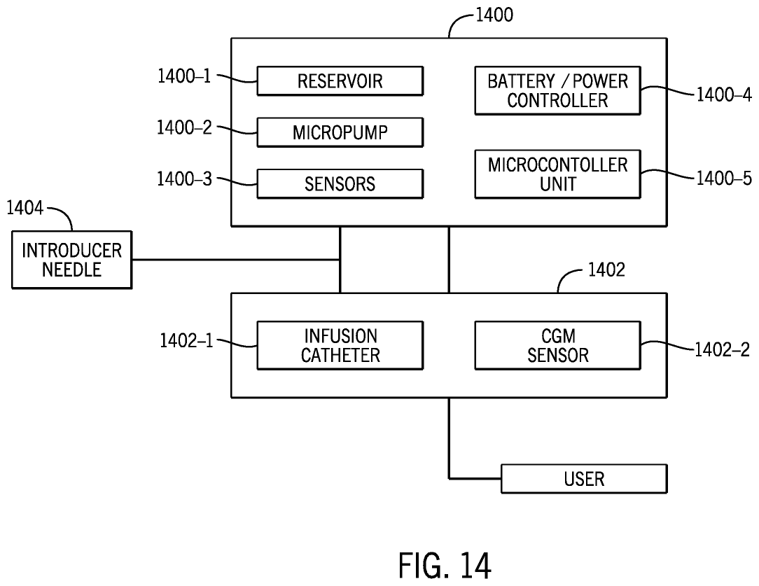

Device 300 also includes reservoir, a microcontroller unit (MCU) (not shown), and a battery and power controller. Cartridge assembly 100 (including integrated infusion catheter and CGM sensor subassembly 102) is a separate component from device 300 (but cartridge assembly 100 may be described as part of device 300 as both the cartridge assembly 100 and device 300 remain on the user in a deployed configuration). The reservoir is configured to receive and store insulin for its delivery over a course of about three days, or as needed. However, reservoir size may be configured for storing any quantity of fluid as required. The micropump fluidly communicates with reservoir to enable infusion as needed. CGM, as known to those skilled in the art, tracks patient glucose levels and permits those levels to be used in algorithms that control flow rate. The micropump may be a MEMS device or other electro-mechanical device known to those skilled in the art. MCU controls the operation of the micropump to deliver insulin through an insulin catheter from the reservoir at specific doses, i.e., flow rates over specified time intervals, based on CGM data converted to desired flow rate via control algorithms. The battery and power controller controls the power to the MCU and the micropump to enable those components to function properly as known to those skilled in the art. The CGM is powered by battery and the power controller through the MCU. (Examples of these components are shown in FIG. 14.)

Cartridge assembly 100 is configured to fit within a cartridge (insertion) compartment opening in the top housing and a channel extending through top housing of device 300. The cartridge (insertion) compartment opening in the top of the housing enables access for the cartridge assembly 100 so it can drive integrated infusion catheter and CGM sensor subassembly 102 into the subcutaneous tissue of a user. Cartridge assembly 100 comprises cartridge housing 100-1, insert or baseplate 100-2 with hole 100-3 to enable subassembly 102 to protrude through and penetrate tissue upon deployment.

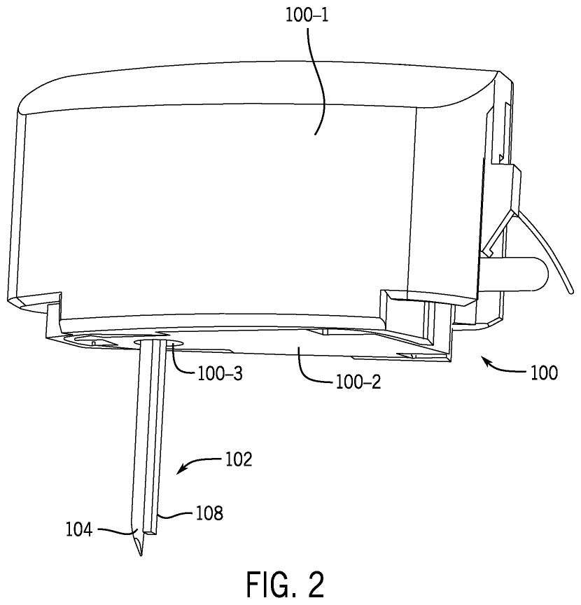

Upon needle insertion, the opening is closed off, so the housing provides some sealing properties and creates a continuous surface as shown in FIG. 2. Detachable activation mechanism 302 compartment is outside of the hermetically sealed compartment that contains the pump and electronics of device 300 for delivering insulin so water ingress can occur without affecting device 104 performance. The amount of water ingress is limited by minimizing the volume of empty space in order to reduce the wetting nuisance that can occur after a user's swim or shower.

In brief, as indicated above, cartridge assembly 100 is configured to move from (1) a telescoping position above the top surface of device 300, wherein integrated infusion needle and CGM sensor subassembly 102 is in a retracted position to (2) an advanced position within device 300, wherein Integrated infusion catheter and CGM sensor subassembly 102 is in a deployed position embedded into the subcutaneous layer of the user's tissue (after insertion). This is described in more detail below.

Integrated infusion catheter and CGM sensor subassembly 102 is part of a closed loop or partially closed loop wearable insulin delivery device such as device 300 driven by blood glucose sensing feedback. Integrating both infusion and CGM sensor capabilities into a single wearable device provides a more discreet and convenient means of optimizing blood glucose levels compared to the currently available commercial options. Combining insertion for CGM and infusion also has the advantages of reducing the number of time users must stick themselves with needles, reduce the number of steps to apply wearable devices and reduce waste associated with insertion mechanisms such as detachable activation mechanism 302.

Subassembly 102 may incorporate a custom designed CGM sensor subassembly or an off the shelf CGM sensor subassembly (commercially available) which includes the sensor chassis, introducer needle, sensor and electrical components or slightly modified versions of these subassemblies.

In FIG. 1, integrated infusion catheter and CGM sensor subassembly 102 incorporates a sharp tipped steel indwelling infusion catheter 104, introducer needle 106 and CGM sensor 108. Cartridge assembly 100 is moved using detachable activation mechanism 304. In short, the introducer needle may be removed by the user or automatically by detachable activation mechanism 304 as known to those skilled in the art. FIG. 2 depicts cartridge assembly 100 after insertion of CGM sensor 108 and after introducer needle 106 has been removed (e.g., manually or automatically). Infusion needle 104 ultimately remains in the subcutaneous tissue for insulin infusion.

FIG. 3 depicts a cross sectional view of the infusion system 302 with cartridge 100 inside detachable activation mechanism 304 in a pre-activation configuration.

FIG. 4 depicts a side view of infusion catheter and CGM sensor subassembly 102 including infusion catheter 104, introducer needle 106 and sensor 108 (not visible) in a pre-activation. End 104a of infusion catheter 104 has a bevel planar shape so that end 104a of infusion catheter 104 is configured and functions as a needle. The bevel surface is oriented relative to introducer needle 106 such that tissue insertion forces bias the infusion catheter 104 against introducer needle 106 in order to keep both needles (catheter 104 and introducer needle 106) in the same insertion hole thereby minimizing scar tissue.

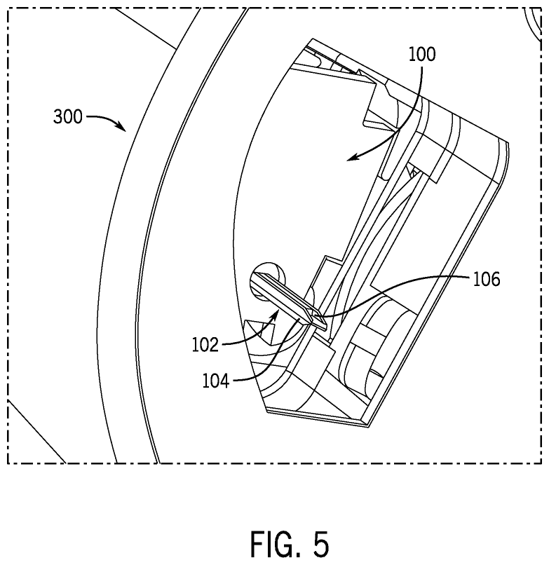

FIG. 5 depicts a close view through an opening in a baseplate of delivery device 300 through which both needles (introducer needle 106 and infusion catheter 104) pass during insertion.

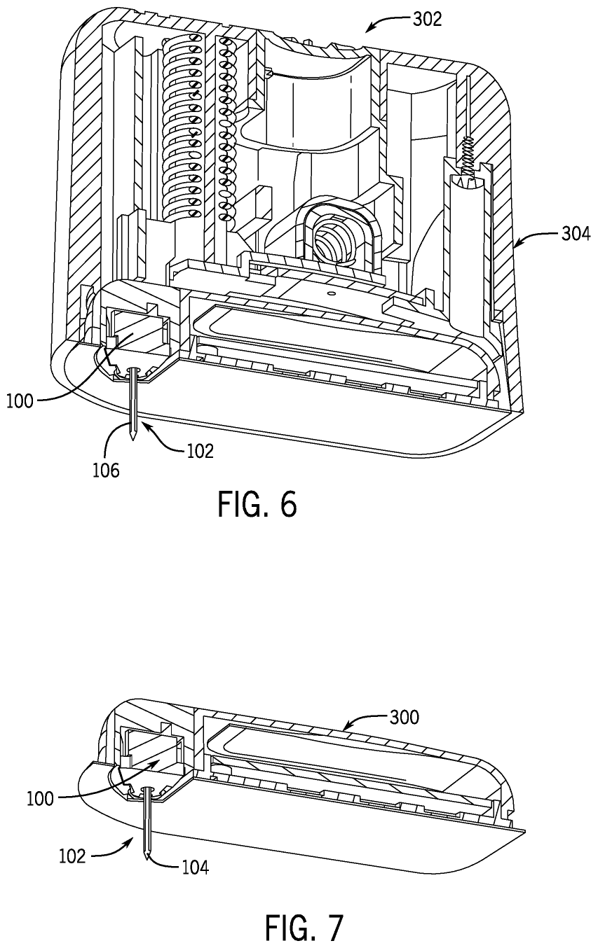

FIG. 6 depicts infusion system 302 after insertion of integrated infusion catheter and CGM sensor subassembly 102 (i.e., infusion catheter 104 and CGM sensor 108) by cartridge 100 deployment (in advance state), but before the introducer needle 106 has been retracted.

FIG. 7 depicts a side cross-sectional view of delivery device 300 in wearable deployed configuration on a user. This view is after detachable activation mechanism 304 has been removed as well as introducer needle 106.

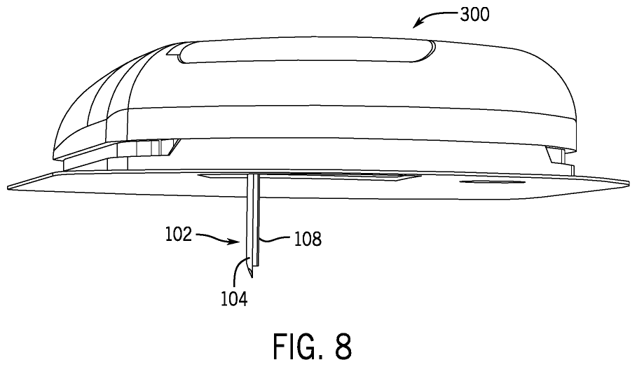

FIG. 8 depicts a side view of delivery device 300 shown in a deployed configuration (applied to a user), but with infusion catheter 104 and CGM sensor 108.

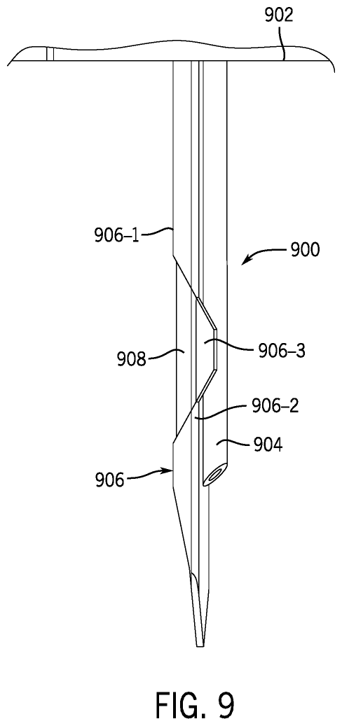

FIG. 9 depicts a side view of another example integrated infusion catheter and CGM sensor subassembly 900 of cartridge assembly 902. In this example, subassembly 900 includes a dull infusion catheter 904 (typically made of steel), introducer needle 906 and CGM sensor 908. Infusion catheter 904 is inserted into tissue using introducer needle 906. Subassembly 900 may be inserted using a C shaped introducer needle or an H shaped introducer needle. H shaped introducer needle 906 has retention elements or features to retain sensor 908 and infusion catheter 904 in place or secure within needle 906. Specifically, in this example, one retention element includes parallel walls 906-1 extending from back wall 906-2 that define a channel on the sensor side to receive and retain the sensor 908 in place (against the introducer needle during insertion), and the other retention element includes opposing walls 906-3 extending from back wall 906-2 that defines a channel on the opposite side thereof to retain and hold infusion catheter 904 in place and against introducer needle 906 as shown. As can be seen, introducer needle 906 has an indented section exposing sensor 908. The distal end of a catheter (steel or otherwise) can be sharp or dull.

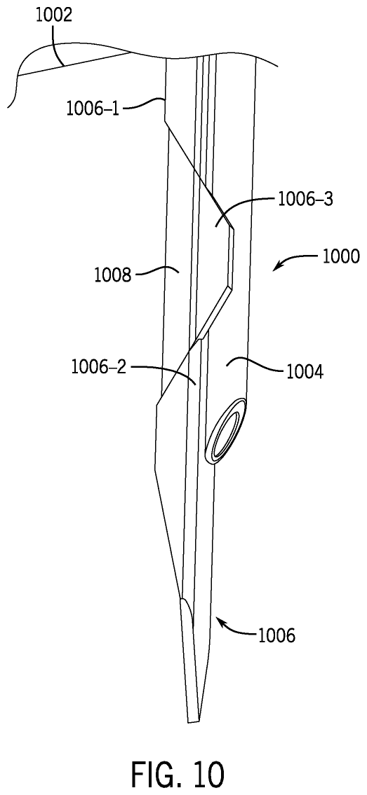

FIG. 10 depicts a side view of another example integrated infusion catheter and CGM sensor subassembly 1000 of cartridge 1002. A single bevel shaped catheter 1004 is oriented so that insertion forces bias it towards introducer (insertion) needle 1006. Thus, catheter 1004 feeds into the hole cut by the H shaped introducer needle 1006. The edges on the inner diameter and outer diameter of catheter 1004 have been etched to dull them (creating by grinding process). The tip is ground to create a slight angle to aid insertion of catheter 1004 into the hole created by introducer needle 1006. The advantage of a steel catheter 1004 that is duller than a hypodermic needle is that it minimizes the wound and wound healing response caused by a sharp that remains in the body for multiple days.

Similar in configuration and functionality as needle 906 above, the H shaped introducer needle 1006 includes retention elements (i.e., parallel walls extending from a back wall) to hold infusion catheter 1004 in one side and CGM sensor 1008 in the other side as shown. Specifically, a retention element includes walls 1006-1 (essentially parallel) extending from back wall 1006-2 that define a channel on the sensor side to receive and retain the sensor 1008 in place (against the introducer needle during insertion), and the other retention element includes opposing walls 1006-3 extending from back wall 1006-2 that defines a channel on the opposite side thereof to retain and hold infusion catheter 1004 in place and against introducer needle 1006 as shown.

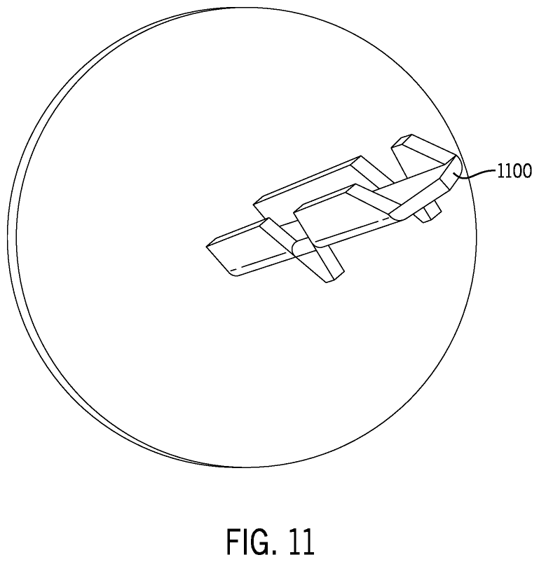

FIG. 11 depicts bottom view another example H shaped introducer needle 1100 (similar to examples above) of an integrated infusion and CGM sensor subassembly extending from a cartridge assembly. Needle 1100 is H shaped made using a sheet metal progressive die fabrication process.

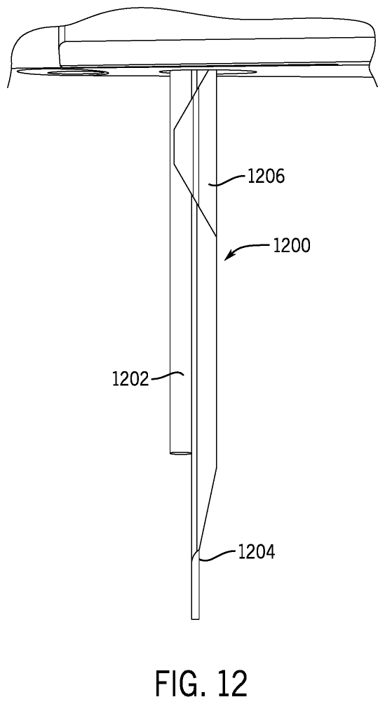

FIG. 12 depicts a side view of another example integrated infusion catheter and CGM sensor subassembly 1200 wherein the distal end of catheter 1202 (steel or plastic including PEEK) is not sharp and does not have any bevel. Similar to the examples above, introducer needle 1204 is H shaped with a retention element or feature on two sides, i.e., parallel walls extending from a back wall, (one of which to hold the position of CGM sensor 1206 and the other retention element or feature, i.e., parallel walls extending from a back wall, to hold the position of catheter 1202 during insertion into the subcutaneous space) that cuts a hole in the tissue through which the sensor 1206 and catheter 1202 pass negating the need for a sharpened distal end to the infusion component (remaining). The avoidance of leaving the sharp in the body during a wear period of the delivery device has the advantage of less discomfort and the creation of less scar tissue.

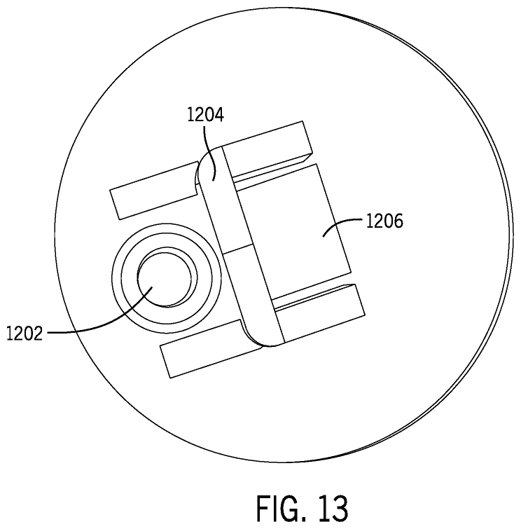

FIG. 13 depicts a view of the same example as that shown in FIG. 12 but from a view normal to the primary axis of catheter 1202.

FIG. 14 depicts a block diagram of example components of (1) device 1400 for delivering insulin and (2) cartridge assembly 1402 of infusion system 100 as described in detail above. Specifically, device 1400 incorporates several components or modules (not shown) in the fluidic pathway including reservoir 1400-1 for storing the insulin, micropump 1400-2 for pumping the insulin, sensors 1400-3 (e.g., pressure) for sensing various parameters in the system and user and tubing connecting infusion needle 1402-1 to reservoir 1400-1 within cartridge assembly 1402. Device 1400 also includes microcontroller unit (MCU) 1400-4 and battery and power controller 1400-5. Cartridge assembly 1402 also includes CGM sensor 1400-5. CGM, as known to those skilled in the art, tracks user glucose levels and permits those levels to be used in algorithms that control flow rate. MCU 1400-4 controls the operation of micropump 1400-2. Integrated infusion catheter 1402-1 and CGM sensor 1402-2 subassembly are shown as separate components in FIG. 14 for illustration purposes, but they are together as they are introduced as described hereinabove. Specifically, infusion catheter 1402-1 and CGM sensor 1402-2 are integrated as shown in the examples above. Introducer needle 1404 is also shown as separate because it is removable but it is part of the integrated subassembly between infusion catheter 1402-1 and CGM sensor 1402-2.

Reservoir 1400-1 is configured to receive and store insulin for its delivery over a course of about three days, or as needed. However, reservoir size may be configured for storing any quantity of fluid as required.

MCU 1400-5 electronically communicates with sensors 1400-3 and micropump 1400-2 as well as the CGM sensor 1402-2, as the monitoring components. Among several functions, MCU 1400-5 operates to control the operation of micropump 1400-2 to deliver insulin through insulin needle 1402-1 from reservoir 1400-1 at specific doses, i.e., flow rates over specified time intervals, based on CGM data converted to desired flow rate via control algorithms.

Battery and power controller 1400-4 controls the power to MCU 1400-5 and micropump 1400-2 to enable those components to function properly as known to those skilled in the art. CGM sensor 1400-2 is powered by battery and power controller 1400-4 through MCU 1400-5.

The components of device 1400 and cartridge assembly 1402 shown in FIG. 14 are only a few components. Those skilled in the art know that device 1400 and cartridge assembly 1402 include additional components.

It is to be understood that the disclosure teaches examples of the illustrative embodiments and that many variations of the invention can easily be devised by those skilled in the art after reading this disclosure and that the scope of the present invention is to be determined by the claims below.

What is claimed is:

1. An infusion system comprising:
a device for delivering a fluid into tissue of a user;
an infusion catheter;
an analyte sensor;
an introducer needle for introducing the infusion catheter and the analyte sensor into the tissue of the user; and
a carrier configure to carry the introducer needle along with the infusion needle and analyte sensor, wherein the carrier is configured to move from (a) first position, wherein the introducer needle, the infusion catheter and the analyte sensor are in a retracted position above the tissue of the user to (b) a second position, wherein the introducer needle, the infusion catheter and the analyte sensor are in a deployed position inserted into tissue of the user.

2. The infusion system of claim 1 wherein the introducer needle is removable.

3. The infusion system of claim 1 wherein the introducer needle includes at least one retention element for holding the infusion catheter or the analyte sensor.

4. The infusion system of claim 3 wherein the retention element includes a back wall and first and second walls extending from the back wall, thereby creating a channel for receiving the analyte sensor or infusion catheter.

5. The infusion system of claim 1 wherein introducer needle is configured to introducer the infusion catheter and analyte sensor into the tissue of the user together.

6. The infusion system of claim 1 further comprising a detachable activation mechanism for causing the cartridge assembly to move from (1) the first position to (2) the second position.

7. The infusion system of claim 1 wherein the fluid is insulin and the analyte sensor is a CGM sensor.

8. An infusion system comprising:
a device for delivering insulin into tissue of a user; and
a cartridge assembly including a subassembly for supporting an infusion needle and a CGM sensor, the subassembly including:
the infusion catheter;
the CGM sensor; and
an introducer needle for introducing the subassembly for supporting the infusion catheter and the CGM sensor into the tissue of the user,
wherein the cartridge assembly configured to move from (a) first position, wherein the subassembly is in a retracted position above the tissue of the user to (b) a second position, wherein the subassembly is in a deployed position inserted into tissue of the user.

9. The infusion system of claim 8 wherein the introducer needle is removable.

10. The infusion system of claim 8 wherein the introducer needle includes at least one retention element for holding the infusion catheter or the CGM sensor.

11. The infusion system of claim 10 wherein the retention element includes a first wall and second and third walls extending from the first wall, thereby creating a channel for receiving the CGM sensor or infusion catheter.

12. The infusion system of claim 8 wherein introducer needle is configured to introducer the infusion catheter and CGM sensor into the tissue of the user simultaneously.

13. The infusion system of claim 8 further comprising a detachable activation mechanism for causing the cartridge assembly to move from (1) the first position to (2) the second position.

14. An infusion system comprising:
a device for delivering insulin into tissue of a user; and
a cartridge assembly including an integrated infusion catheter and CGM sensor subassembly and an introducer needle for introducing the integrated infusion catheter and CGM sensor subassembly into the tissue of the user,
wherein the cartridge assembly configured to move from (a) first position, wherein the integrated infusion catheter and CGM sensor subassembly is in a first position above the tissue of the user to (b) a second position, wherein the integrated infusion catheter and CGM sensor subassembly is in a deployed position inserted into tissue of the user.

15. The infusion system of claim 14 the subassembly includes an infusion catheter and a CGM sensor.

16. The infusion system of claim 15 wherein the introducer needle is removable.

17. The infusion system of claim 16 wherein the introducer needle includes at least one retention element for holding the infusion catheter or the CGM sensor.

18. The infusion system of claim 17 wherein the retention element includes a back wall and first and second walls extending from the back wall, thereby creating a channel for receiving the CGM sensor or infusion catheter.

19. The infusion system of claim 15 wherein introducer needle is configured to introduce the infusion catheter and CGM sensor into the tissue of the user together.

20. The infusion system of claim 14 further comprising a detachable activation mechanism for causing the cartridge assembly to move from (1) the first position to (2) the second position.
